(19) United States
(12) Patent Application Publication
(10) Pub. No.: US 2023/0293875 A1
(43) Pub. Date: Sep. 21, 2023

(54) Integrated Catheter with Manually-Actuable Valve Mechanism

(71) Applicant: Becton, Dickinson and Company, Franklin Lakes, NJ (US)

(72) Inventors: Erica E. Neumann, Sandy, UT (US); Hadley Anne Hendrick, Pasadena, CA (US); John M. Lackey, West Valley City, UT (US); Jonathan Karl Burkholz, Salt Lake City, UT (US); Ralph L. Sonderegger, Farmington, UT (US); Shaun Staley, Murray, UT (US); Weston F. Harding, Lehi, UT (US)

(21) Appl. No.: 18/122,982

(22) Filed: Mar. 17, 2023

Related U.S. Application Data
(60) Provisional application No. 63/321,945, filed on Mar. 21, 2022.

(51) Int. Cl. A61M 39/22; A61M 39/10

(52) U.S. Cl. CPC A61M 39/223; A61M 39/10; A61M 2039/1077; A61M 2039/224; A61M 2039/229

(57) ABSTRACT

Disclosed herein is an intravascular catheter assembly, including a catheter adapter having a distal end; a proximal end; a first lumen arranged between and in fluid communication with the distal end and the proximal end; a catheter arranged at the distal end of the catheter adapter and in fluid communication with the lumen; and a first side port in fluid communication with the lumen; a first fluid conduit having a distal end and a proximal end, the distal end of the fluid conduit coupled to the side port; and a connector having a proximal end, a distal end connected to the fluid conduit, and a second lumen therethrough, the connector comprising a manually-actuable valve.

### CROSS-REFERENCE TO RELATED APPLICATION

The present application claims priority to U.S. Provisional Application Ser. No. 63/321,945, entitled “Integrated Catheter with Manually-Actuable Valve Mechanism”, filed Mar. 21, 2022, the entire disclosure of which is hereby incorporated by reference in its entirety.

### BACKGROUND OF THE INVENTION

### Field of the Invention

The present disclosure relates generally to intravenous (IV) catheter assemblies and, more specifically, to IV catheter assemblies with manually-actuable valve mechanisms to stop flow through the catheter adapter and/or any attached extension sets and allow for replacement of various components.

### Description of Related Art

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of intravenous (IV) catheter device includes a catheter that is over-the-needle. As its name implies, the catheter that is over-the-needle may be mounted over an introducer needle having a sharp distal tip. The IV catheter device may include a catheter adapter, the catheter extending distally from the catheter adapter, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

IV catheter devices can include an extension set with a plurality of ports and/or connectors for administering or withdrawing fluids from the patient, and/or flushing the catheter device. One or more of these ports and/or connectors may be replaceable; however, there is currently no way to occlude the extension set during such a replacement procedure. Thus, a need exists in the art for an IV catheter device that allows for simple occlusion of tubing associated therewith.

### SUMMARY OF THE INVENTION

Provided herein is an intravascular catheter assembly, including a catheter adapter having a distal end, a proximal end, a first lumen arranged between and in fluid communication with the distal end and the proximal end, a catheter arranged at the distal end of the catheter adapter and in fluid communication with the lumen, and a first side port in fluid communication with the lumen, with a first fluid conduit having a distal end and a proximal end. The distal end of the fluid conduit may be coupled to the side port and a connector having a proximal end, a distal end connected to the fluid conduit, and a second lumen therethrough, the connector including a manually-actuable valve.

In certain configurations, the valve is arranged at the proximal end of the connector. Optionally, the valve may be arranged at the distal end of the connector. The valve may include a sliding mechanism configured to displace a portion of the second lumen, such that the portion of the second lumen has a longitudinal axis that is offset from a longitudinal axis of the second lumen. The connecter may be a t-connector. Optionally, the connector may be a y-connector.

In additional configurations, the assembly may also include a second fluid conduit extending from the connector. In further configurations, the assembly also includes a needleless access connector coupled to the connecter, wherein the valve is rotatable between at least a first position in which the valve obstructs fluid flow to the needleless access connector, a second position in which the valve obstructs fluid flow to the second fluid conduit, and a third position in which fluid can flow through the valve to both the needles access connector and the second fluid conduit.

The second lumen of the assembly may have an inner diameter substantially equivalent to an inner diameter of the first fluid conduit and the second fluid conduit. Optionally, the valve may be rotatable around an axis defined by the first fluid conduit. In certain configurations, the valve may include a stopcock. The stopcock may be a one-way stopcock. In other configurations, the stopcock may be a three-way stopcock. Optionally, the stopcock may include one or more arms. The stopcock may be rotatable about an axis normal to a flow of fluid through the second lumen.

In other configurations, the connector of the assembly may also include a second side port in fluid communication with the second lumen. Optionally, the stopcock may be rotatable about an axis defined by the second side port. In still other configurations, the assembly may include a needleless access connector coupled to the connector. The needleless access connector may be releasably coupled to the connector. In still other configurations, the valve may be rotatable between at least a first position in which the valve obstructs fluid flow through the second lumen, and a second position in which fluid can flow through the lumen. The valve, when in the first position, engages with the needleless access connector, thereby preventing removal of the needleless access connector.

In still other configurations, the intravascular catheter assembly includes a catheter adapter having a distal end, a proximal end, a first lumen arranged between and in fluid communication with the distal end and the proximal end, a catheter arranged at the distal end of the catheter adapter and in fluid communication with the lumen, and a first side port in fluid communication with the lumen. The intravascular catheter assembly further includes a first fluid conduit having a distal end and a proximal end, the distal end of the fluid conduit coupled to the side port, a connector comprising a proximal end, a distal end connected to the fluid conduit, and a second lumen therethrough, and a needleless access connector coupled to the connecter. At least one of the connector and the needleless access connector includes a valve that is rotatable between at least a first position in which the valve obstructs fluid flow to the needleless access connector, a second position in which the valve obstructs fluid flow to the second fluid conduit, and a third position in which fluid can flow through the valve to both the needles access connector and the second fluid conduit.

### BRIEF DESCRIPTION OF THE DRAWINGS

FIG. 1 is a perspective view of a catheter assembly according to non-limiting embodiments as described herein;

FIGS. 2A-2H are cross-sectional views of a catheter assembly according to non-limiting embodiments as described herein;

FIG. 3 is a perspective view of a catheter assembly according to non-limiting embodiments as described herein;

FIGS. 4A-4D are cross-sectional views of a catheter assembly according to non-limiting embodiments as described herein;

FIGS. 5A-5C are perspective views of a catheter assembly according to non-limiting embodiments as described herein;

FIGS. 6A-6F are cross-sectional views of a catheter assembly according to non-limiting embodiments as described herein;

FIG. 7 is a perspective view of a catheter assembly according to non-limiting embodiments as described herein;

FIGS. 8A-8E are cross-sectional (FIGS. 8A-8D) and perspective (FIG. 8E) views of a catheter assembly according to non-limiting embodiments as described herein; and

FIGS. 9A-9B are perspective views of a catheter assembly according to non-limiting embodiments as described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

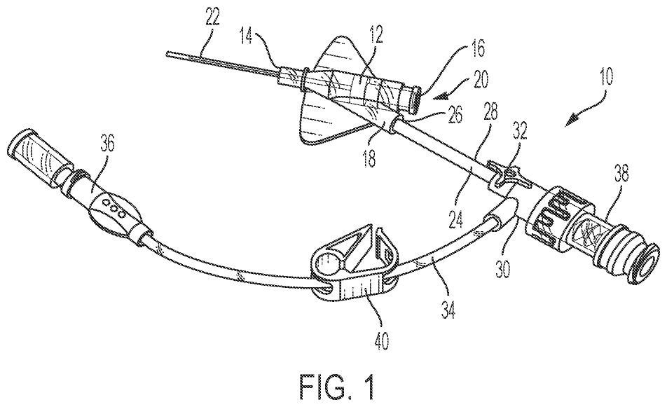

Referring now to FIG. 1, shown is a non-limiting embodiment of a catheter assembly 10, which may include a catheter adapter 12, which may include a distal end 14 and a proximal end 16. In some embodiments, the catheter adapter 12 may include a side port 18 disposed between the distal end 14 and a proximal end 16. In some embodiments, the first catheter adapter 12 may include a first lumen 20 extending through the distal end 14 and the proximal end 16.

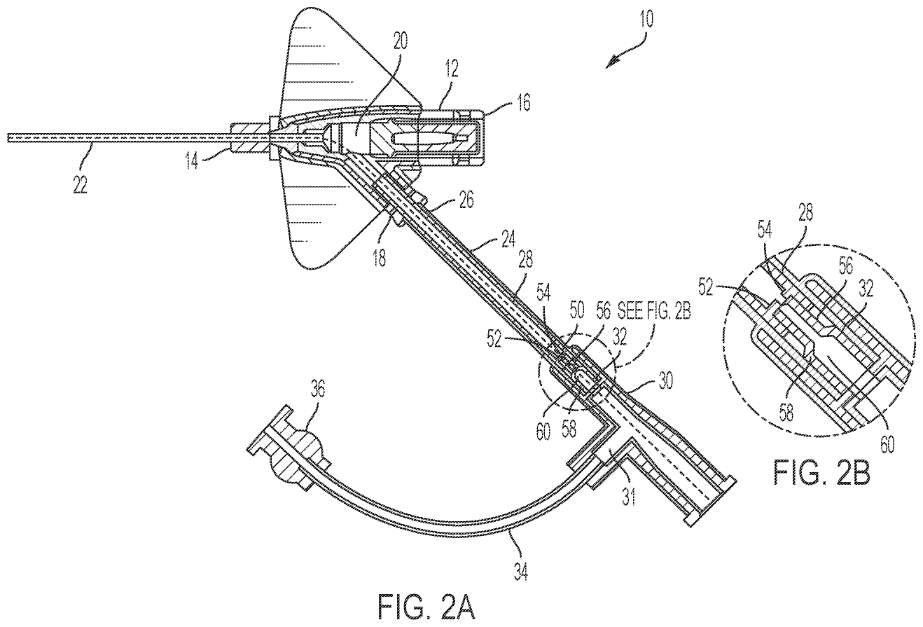

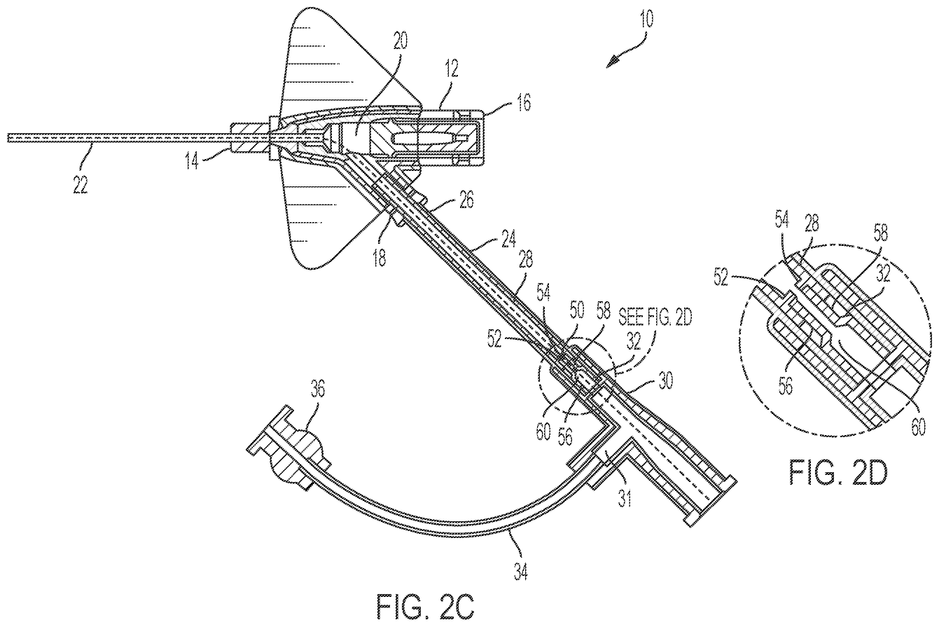

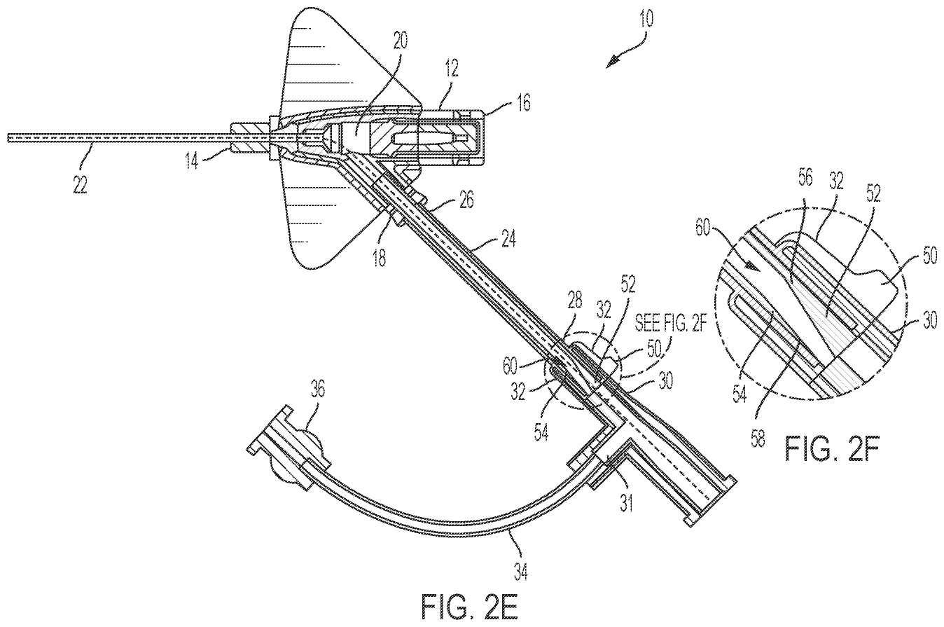

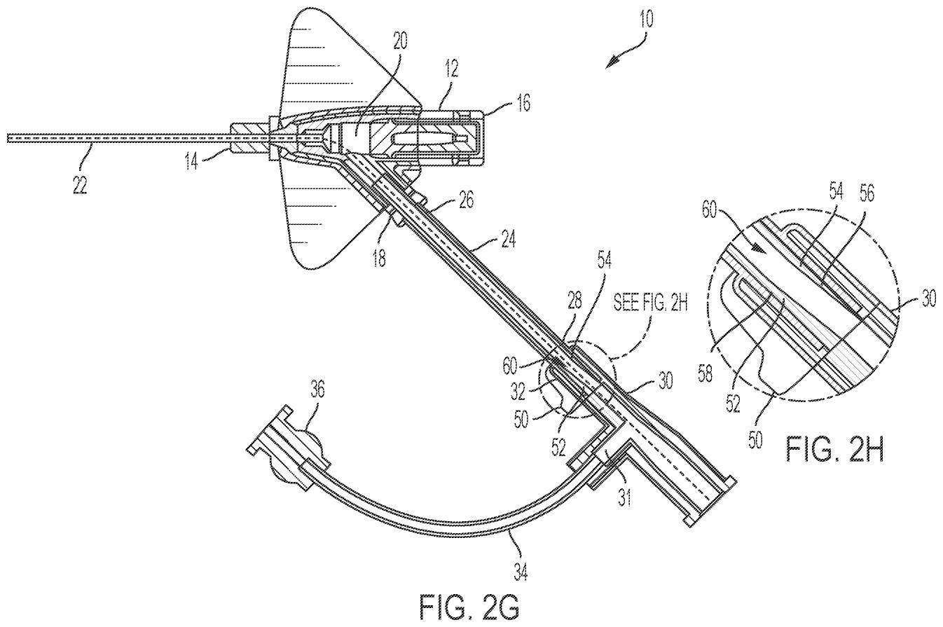

In some non-limiting embodiments or aspects, the first catheter assembly 10 may include a first catheter 22 extending from the distal end 14. In some embodiments, the first catheter 22 may include a peripheral intravenous catheter, a midline catheter, or a peripherally-inserted central catheter. Catheter 22 may be formed of any suitable material and may be of any useful length, as known to those of skill in the art. In some non-limiting embodiments or aspects, the first catheter assembly 10 may include a first fluid conduit 24 extending from the side port 18. First fluid conduit 24 may be formed of any suitable material known to those of skill in the art, and may have a distal end 26 and a proximal end 28, and first fluid conduit 24 may be coupled, at distal end 26 thereof, to side port 18. In some non-limiting embodiments or aspects, a connector 30 may be coupled to a proximal end 28 of first fluid conduit 24. Connector 30 may be a t-connector (e.g., one side port arranged at a 90 degree angle relative to a longitudinal axis of connector 30), a y-connector (e.g., one side port arranged at an angle of about 15 to about 75 degrees, optionally a 25, 60, or 75 degree angle, relative to a longitudinal axis of connector 30), or any other type of connector known in the art, and may include a second lumen 31 (shown in FIG. 2A) therethrough, having any number of branches suitable for the type of connector.

In some non-limiting embodiments or aspects, catheter assembly 10 may include an extension set including a second fluid conduit 34. In non-limiting embodiments, connector 30 includes a side port to which extension set (e.g., second fluid conduit 34) may be connected. Extension sets are known to those of skill in the art and are commercially available from, for example, Becton, Dickinson and Company under the tradename NEUTRACLEAR. In some non-limiting embodiments or aspects, second fluid conduit 34 may include a luer connection 36 at an end thereof. In some non-limiting embodiments or aspects, the extension set may include a clamp 40, to allow for occlusion of second fluid conduit 34. Clamp 40 and second fluid conduit 34 may be formed of any suitable materials known to those of skill in the art. In non-limiting embodiments, second lumen 31 has an inner diameter that is substantially equivalent to an inner diameter of first fluid conduit 24 and/or second fluid conduit 34.

Catheter assembly 10 may include manually-actuable valve 32 for slowing and/or stopping fluid flow, for example through first fluid conduit 24, between first fluid conduit 24 and connector 30, and/or between connector 30 and a second fluid conduit 34, by occluding flow. As used herein, “occlude” or “occlusion” means at least a slowing of fluid flow through a conduit, such as fluid conduit 24, and includes within its scope complete cessation of flow through an occluded portion of a conduit. While the accompanying drawings may show valve 32 as part of, or separate from, connector 30, those of skill will appreciate that useful valves as described herein can be arranged, relative to connector 30, in any suitable manner to occlude flow to one or both of a needleless access connector 38 and/or a second fluid conduit 34.

Those of skill will appreciate that arrangements of manually-actuable valve 32 along fluid conduit 24 and/or as part of connector 30 can allow for cessation of flow of fluid, such as blood, from first lumen 20 to connector 30 and/or second fluid conduit 34. This can allow a user, such as a healthcare professional, to replace one or more components of catheter assembly 10, such as needleless access connector 38. Needleless access connectors are known to those of skill in the art and are commercially available from, for example, Becton, Dickinson and Company under the tradenames MAXPLUS, MAXZERO, SMARTSITE, and Q-SYTE. Manually-actuable valve 32 can assume any useful configuration, for example those as described and illustrated herein. That is, while certain exemplary figures may depict manually-actuable valve 32 as a stopcock, sliding mechanism, or the like, it is to be appreciated that any manually-actuable valve 32 shown in any accompanying figure may be utilized in any other embodiment shown and described herein.

In non-limiting embodiments, manually-actuable valve 32 is part of connector 30, which may connect first fluid conduit 24 to second fluid conduit 34 and/or to needleless access connector 38. Manually-actuable valve 32 may be arranged at a distal end of connector 30, a proximal end of connector 30, or between a proximal end and distal end of connector 30.

Turning to FIGS. 2A-2D, shown is a non-limiting embodiment of a catheter assembly 10, in which manually-actuable valve 32 is a rotatable valve. While shown as being rotatable about an axis defined by first fluid conduit 24 and connector 30, those of skill will appreciate that manually-actuable valves, such as stopcocks described herein, can be arranged to rotate about any axis, so long as fluid flow through the valve can be slowed or stopped. Additionally, stopcocks as described herein may include one or more arms for grasping by a user, and may be one-way stopcocks (e.g., one open position), or three-way stopcocks (e.g., more than one open position), such that rotating a stopcock 90, 180, or 270 degrees can provide differing fluid flows.

As shown in FIGS. 2A-2D, first fluid conduit 24 may include at proximal end 28 thereof, first and second protrusions 50, 52, narrowing an inner diameter of first fluid conduit 24 at first passage 54. First passage 54 may be centered within first fluid flow conduit 24, or may be offset from center. As also shown, manually-actuable valve 32 may include first and second walls 56, 58, defining a second passage 60. One or both of first and second walls 56, 58 may be angled relative to second passage 60, for example as shown in FIGS. 2A-2D. As shown in FIGS. 2A-2B, in a first configuration, manually-actuable valve 32 is closed, as first wall 56 is angled such that first passage 54 is offset from second passage 60. Manually-actuable valve 32 may be rotated about an axis defined by first fluid conduit 24 to a second configuration as shown in FIGS. 2C-2D, where first wall 56 no longer occludes flow through first passage 54, such that first passage 54 and second passage 60 are in fluid communication, and fluid can pass from first fluid conduit 24 to connector 30.

Turning to FIGS. 2E-2H, shown is another embodiment in which manually-actuable valve 32 is arranged at distal end of connector 30, and includes first wall 56 and second wall 58. As shown in FIGS. 2E-2F, first wall 56 and second wall 58 are arranged such that when manually-actuable valve 32 is in a first configuration, flow through second passage 60 to second lumen 31 within connector 30 is obstructed due to the arrangement of the walls relative to connector 30. As shown in FIGS. 2G-2H, when manually-actuable valve is rotated about an axis defined by first fluid conduit 24 to a second configuration, the arrangement of first and second walls 56, 58 is modified, such that second passage 60 is aligned with second lumen 31 and fluid may flow therethrough. In the embodiments shown in FIGS. 2A-2H, manually-actuable valve 32 may include any protrusions, e.g., protrusion 50, or surface modifications to allow for a user to grasp and rotate the valve.

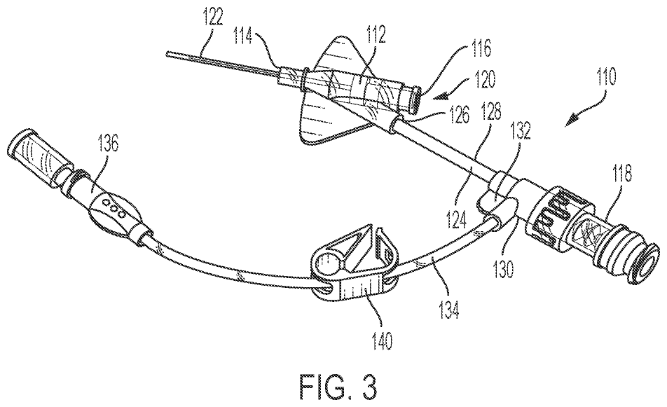

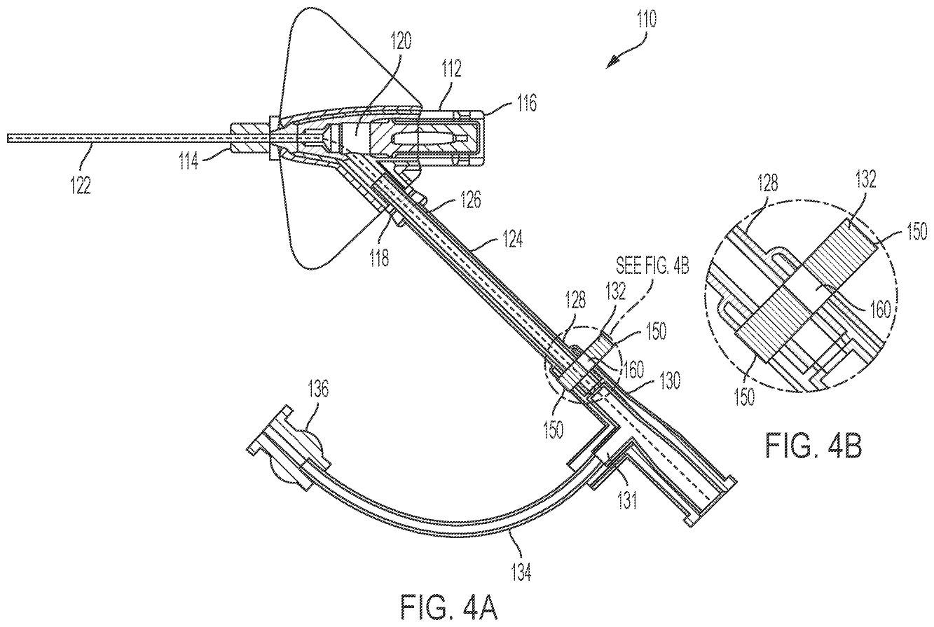

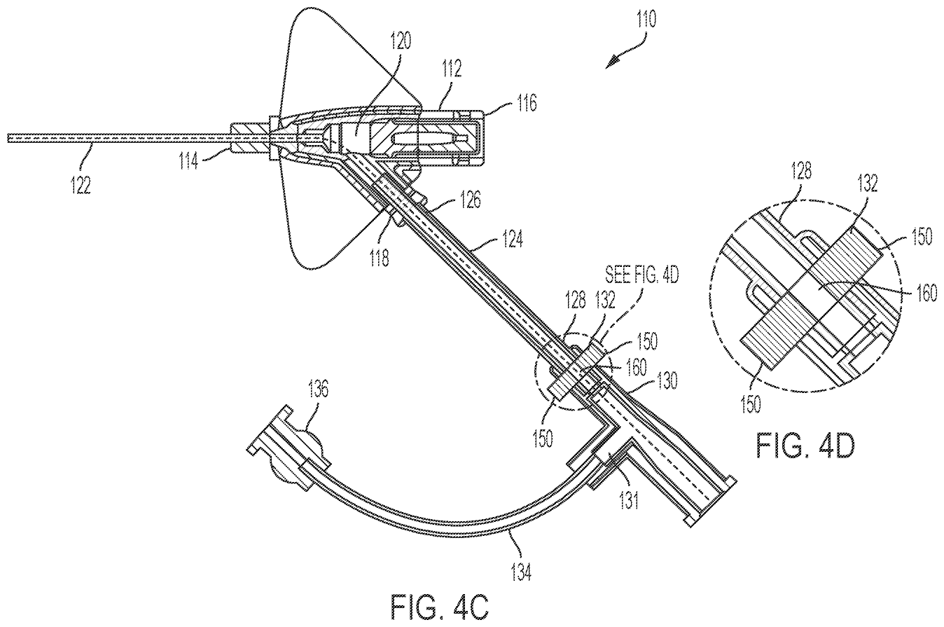

Turning to FIGS. 3-4D, shown is a non-limiting embodiment of a catheter assembly 110, which may include components as described herein above. In the illustrated non-limiting embodiment, manually-actuable valve is configured as a slidable occlusion mechanism 132 including a second passage 160. As shown in FIGS. 4A-4B, in a first configuration, second passage 160 is offset from first fluid conduit 124 and second lumen 131 of connector 130. By pressing on contact surface 150, a user can move the slidable occlusion mechanism 132 between first configuration, where second passage 160 is offset as described above, to a second configuration (FIGS. 4C-4D), where second passage 160 is in fluid communication with first fluid conduit 124 and second lumen 131 of connector 130. Contact surface 150 may include any protrusions or surface modifications to allow for a user to actuate the slidable mechanism.

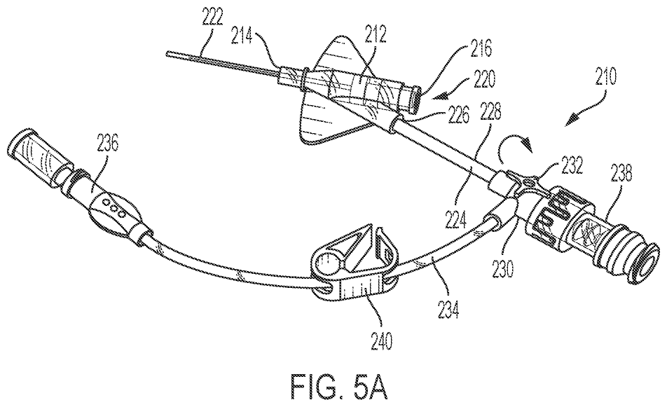

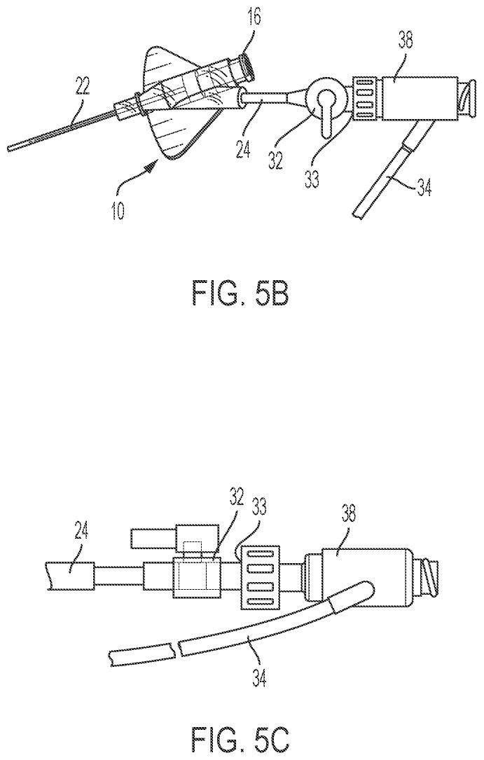

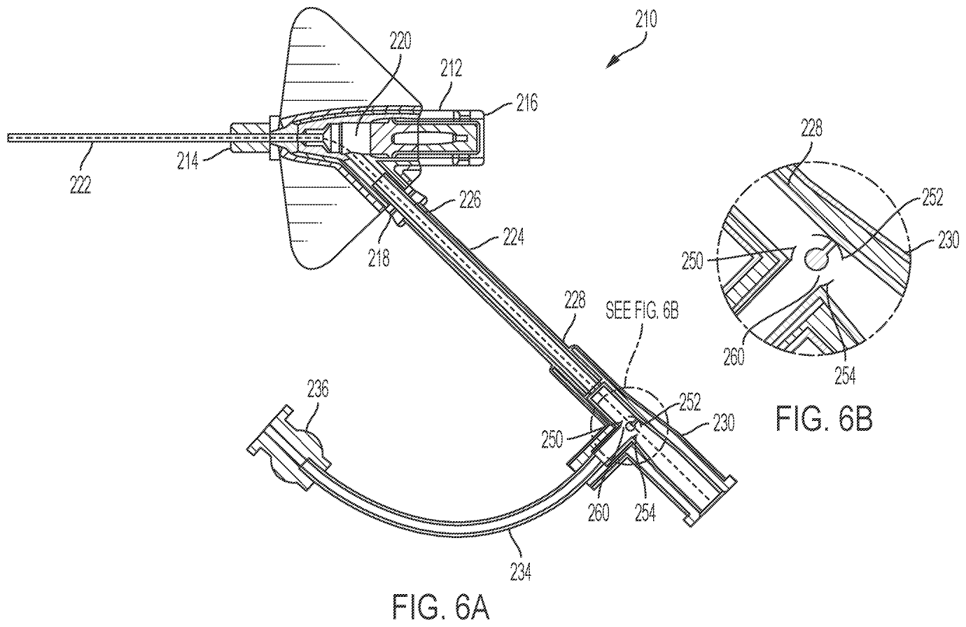

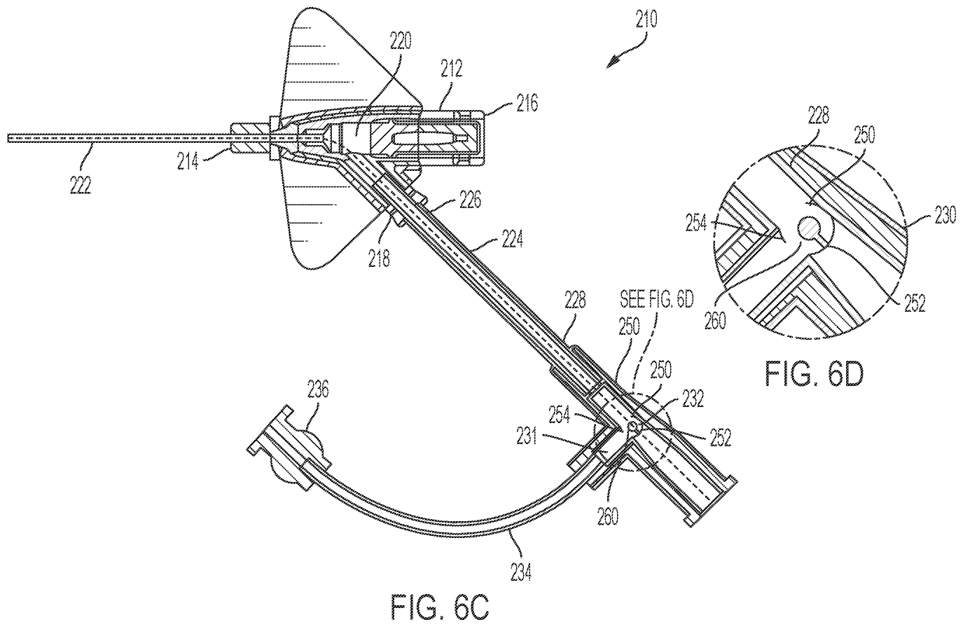

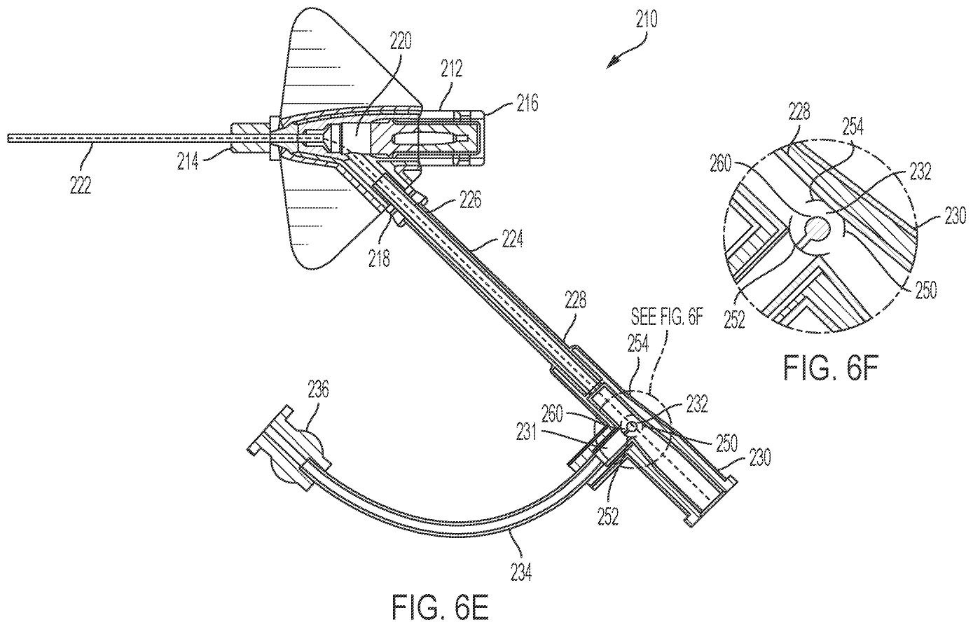

Turning to FIGS. 5A-6F, shown is a non-limiting embodiment of a catheter assembly 210, which may include components as described herein above. In the illustrated non-limiting embodiment, manually-actuable valve is configured as a stopcock 232, optionally arranged as part of connector 230. For example, FIGS. 5A and 6A-6F show an embodiment where stopcock 232 is integral with connector 230. Alternatively, for example as shown in FIGS. 5B-5C, a manually-actuable valve 32, such as a stopcock as described herein, may be included as a separate component from any connector, for example upstream (e.g., closer to catheter assembly 10). In non-limiting embodiments, manually-actuable valve 32 may include a luer connection 33 to allow reversible coupling between manually-actuable valve 32 and one or more components of a catheter assembly 10, such as a connector or needleless access connector 38, such as shown in FIGS. 5B-5C.

Returning to FIGS. 5A and 6A-6F, stopcock 232 may be rotatable about any suitable axis of connector 230. In non-limiting embodiments, stopcock 232 is rotatable about an axis normal to a flow of fluid through second lumen 231. As shown in FIGS. 6A-6F, stopcock 232 may include first wall 250, second wall 252, and/or third wall 254. In non-limiting embodiments, first, second, and/or third walls 250, 252, 254 can define one or more passages 260 therethrough, such that depending on the rotational position of stopcock 232, fluid flow from first fluid conduit 224 through connector 230 may be occluded, or may be open to needleless connector 238 and/or second fluid conduit 234. For example, as shown in FIGS. 6A-6B, stopcock 232 is arranged in a first configuration, where fluid from first fluid conduit 224 is open to connector 230 and second fluid conduit 234. In FIGS. 6C-6D, stopcock 232 has been rotated, and is arranged in a second configuration, where flow from first fluid conduit 224 is occluded from connector 230, but is open to second fluid conduit 234. In FIGS. 6E-6F, stopcock 232 is arranged in a third configuration, where flow from first fluid conduit 224 is occluded from second fluid conduit 234, but is open to connector 230.

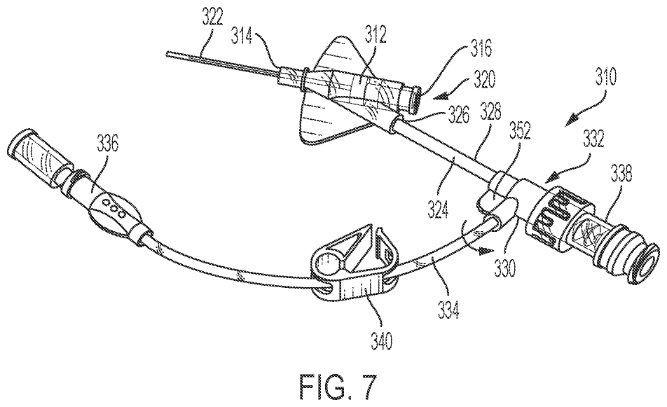

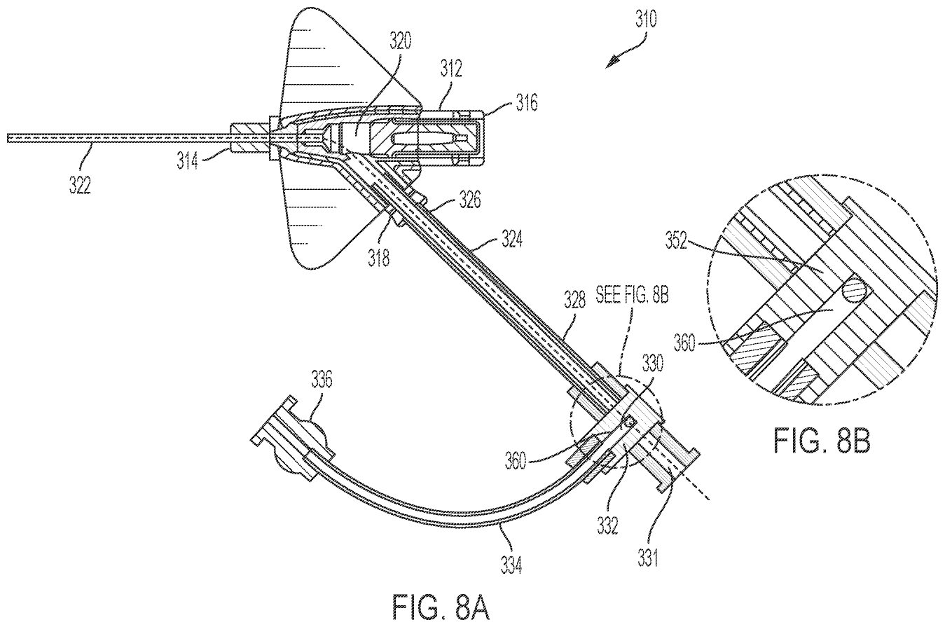

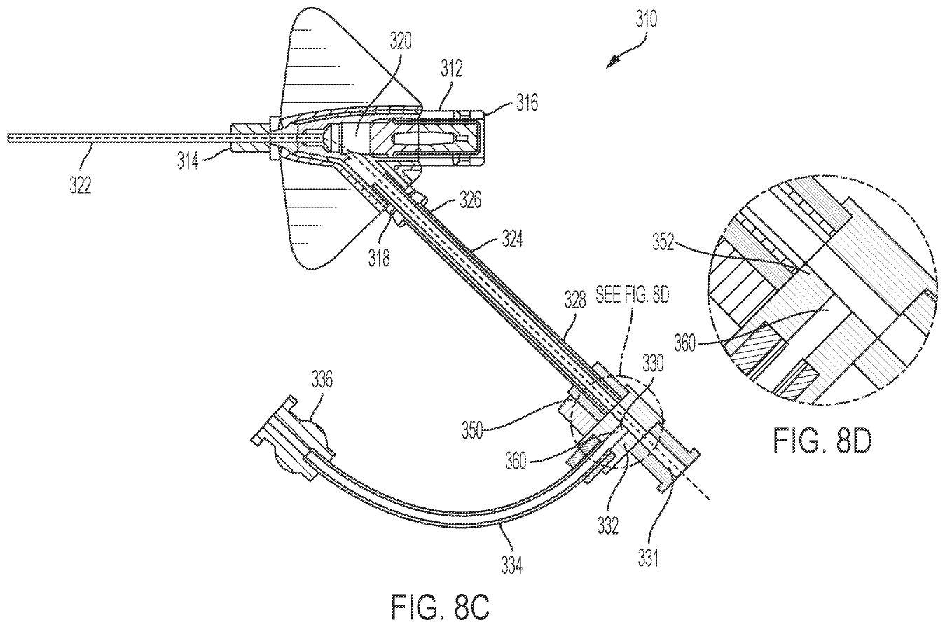

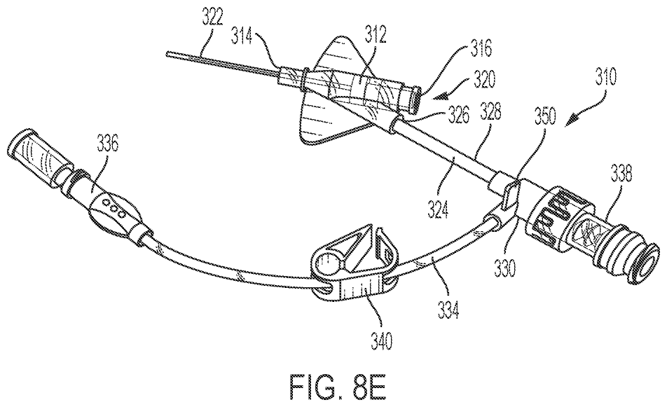

Turning to FIGS. 7-8E, shown is a non-limiting embodiment of a catheter assembly 310, which may include components as described herein above. In the illustrated non-limiting embodiment, manually-actuable valve 332 is configured at a junction of connector 330 and second fluid conduit 334, and is rotatable about an axis defined by second fluid conduit 334 as shown in FIGS. 7 and 8E. Manually-actuable valve 332 includes passage 360 that may be in fluid communication with second fluid conduit 334 and second lumen 331 of connector 330. Manually-actuable valve 332 may also include a wall 352 at an end of passage 360. As shown in FIGS. 8A-8B, in a first configuration of manually-actuable valve 332, wall 352 occludes passage 360, such that fluid cannot flow from first fluid conduit 324 to second fluid conduit 334 or to second lumen 331 of connector 330. In this way, manually-actuable valve 332 can be placed into a closed position to allow for replacement of, for example, a needleless access connector 338 arranged at a proximal end of connector 330 and/or a medical device attached to luer 326. As shown in FIGS. 8C-8E, in a second configuration of manually-actuable valve 332, wall 352 is displaced, opening passage 360 to second lumen 331 of connector 330, thus allowing flow from first fluid conduit 324 to second fluid conduit 334 and to, for example, a needleless access connector 338 arranged at a proximal end of connector 330. Those of skill in the art will appreciate that open and closed configurations of passage 360 may be associated with arrangement of valve 332 in either position illustrated in FIG. 7 or 8E. In the embodiments shown in FIGS. 8A-8E, manually-actuable valve may include any protrusions, e.g., protrusion 350, or surface modifications to allow for a user to grasp and rotate the valve 332.

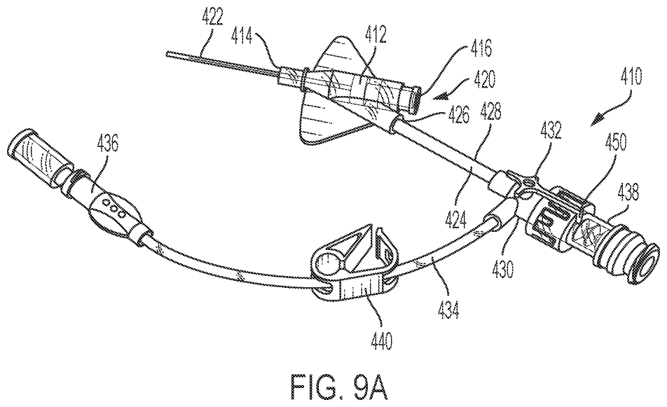

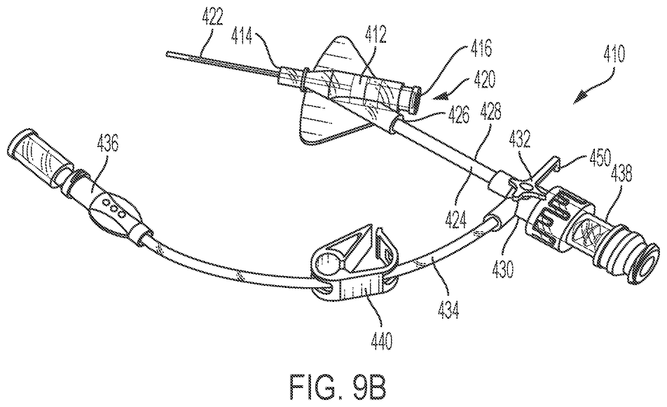

Turning to FIGS. 9A-9B, shown is a non-limiting embodiment of a catheter assembly 410, which may include components as described herein above. In the illustrated non-limiting embodiment, manually-actuable valve may be a stopcock 432 configured to occlude and/or open flow between first fluid conduit 424 and connector 430 and/or second fluid conduit 434 as described previously. In non-limiting embodiments, stopcock 432 includes a flange 450. Flange 450 may be configured such that, in certain configurations of stopcock 432, flange 450 may reversibly couple with needleless access connector 438. In non-limiting embodiments, in a first configuration, for example as shown in FIG. 9A, flange 450 is coupled to needleless access connector 438, and fluid flow between first fluid conduit 424 and needleless access connector 438 is open. The coupling of flange 450 and needleless access connector 438 prevents removal of needleless access connector 438, and thus eliminates accidental disconnection, and loss of fluid flowing from first fluid conduit 424 through connector 430 to needleless access connector 438. In non-limiting embodiments, in a second configuration, for example as shown in FIG. 9B, flange 450 is not coupled to needleless access connector 438, and fluid flow between first fluid conduit 424 and needleless access connector 438 is occluded. In such a second configuration, needleless access connector 438 can be removed from connector 430, but because of the orientation of stopcock 432, no fluid will flow through connector 430 to any port to which needleless access connector 438 can be attached.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. An intravascular catheter assembly, comprising:
a catheter adapter comprising:
a distal end;
a proximal end;
a first lumen arranged between and in fluid communication with the distal end and the proximal end;
a catheter arranged at the distal end of the catheter adapter and in fluid communication with the lumen; and
a first side port in fluid communication with the lumen;
a first fluid conduit having a distal end and a proximal end, the distal end of the fluid conduit coupled to the side port; and
a connector comprising a proximal end, a distal end connected to the fluid conduit, and a second lumen therethrough, the connector comprising an actuable valve.

2. The intravascular catheter assembly of claim 1, wherein the valve is arranged at the proximal end of the connector.

3. The intravascular catheter assembly of claim 1, wherein the valve is arranged at the distal end of the connector.

4. The intravascular catheter assembly of claim 1, wherein the valve comprises a sliding mechanism configured to displace a portion of the second lumen, such that the portion of the second lumen has a longitudinal axis that is offset from a longitudinal axis of the second lumen.

5. The intravascular catheter assembly of claim 1, wherein the connecter is at least one of a t-connector and a y-connector.

6. The intravascular catheter assembly of claim 1, wherein the valve is at least one of manually and remotely actuatable.

7. The intravascular catheter assembly according to claim 5, further comprising a second fluid conduit extending from the connector.

8. The intravascular catheter assembly according to claim 7, further comprising a needleless access connector coupled to the connecter, wherein:
the valve is rotatable between at least a first position in which the valve obstructs fluid flow to the needleless access connector, a second position in which the valve obstructs fluid flow to the second fluid conduit, and a third position in which fluid can flow through the valve to both the needles access connector and the second fluid conduit.

9. The intravascular catheter assembly according to claim 7, wherein the second lumen has an inner diameter substantially equivalent to an inner diameter of the first fluid conduit and the second fluid conduit.

10. The intravascular catheter assembly of claim 5, wherein the valve is rotatable around an axis defined by the first fluid conduit.

11. The intravascular catheter assembly of claim 5, wherein the valve comprises a stopcock.

12. The intravascular catheter assembly of claim 11, wherein the stopcock is a one-way stopcock.

13. The intravascular catheter assembly of claim 11, wherein the stopcock is a three-way stopcock.

14. The intravascular catheter assembly of claim 11, wherein the stopcock comprises one or more arms.

15. The intravascular catheter assembly of claim 11, wherein the stopcock is rotatable about an axis normal to a flow of fluid through the second lumen.

16. The intravascular catheter assembly of claim 11, wherein the connector further comprises a second side port in fluid communication with the second lumen.

17. The intravascular catheter assembly of claim 16, wherein the stopcock is rotatable about an axis defined by the second side port.

18. The intravascular catheter assembly of claim 1, further comprising a needleless access connector coupled to the connector.

19. The intravascular catheter assembly of claim 18, wherein:
the valve is rotatable between at least a first position in which the valve obstructs fluid flow through the second lumen, and a second position in which fluid can flow through the lumen; and
the valve, when in the first position, engages with the needleless access connector, thereby preventing removal of the needleless access connector.

20. An intravascular catheter assembly, comprising:
a catheter adapter comprising:
a distal end;
a proximal end;
a first lumen arranged between and in fluid communication with the distal end and the proximal end;
a catheter arranged at the distal end of the catheter adapter and in fluid communication with the lumen; and
a first side port in fluid communication with the lumen;
a first fluid conduit having a distal end and a proximal end, the distal end of the fluid conduit coupled to the side port,
a connector comprising a proximal end, a distal end connected to the fluid conduit, and a second lumen therethrough; and
a needleless access connector coupled to the connecter, wherein at least one of the connector and the needleless access connector comprises a valve that is rotatable between at least a first position in which the valve obstructs fluid flow to the needleless access connector, a second position in which the valve obstructs fluid flow to the second fluid conduit, and a third position in which fluid can flow through the valve to both the needles access connector and the second fluid conduit.
